(19) United States
(12) Patent Application Publication
(10) Pub. No.: US 2026/0183515 A1
(43) Pub. Date: Jul. 2, 2026

(54) CATHETER STEERING SWASHPLATE WITH PULLEY SYSTEM

(71) Applicant: yoR Labs, Inc., Beaverton, OR (US)

(72) Inventor: Spencer Theodore Slam, Portland, OR (US)

(21) Appl. No.: 19/126,567

(22) PCT Filed: Nov. 6, 2023
(86) PCT No.: PCT/US2023/078863

§ 371 Date: May 1, 2025

Related U.S. Application Data
(60) Provisional application No. 63/382,773, filed on Nov. 8, 2022.

(51) Int. Cl. A61M 25/01; A61B 8/00; A61B 8/12

(52) U.S. Cl. CPC A61M 25/0147; A61B 8/12; A61B 8/445; A61M 2025/015

(57) ABSTRACT

Steering mechanisms and assemblies for steering the distal end of a catheter are disclosed herein. A catheter steering mechanism can include a spindle comprising one or more spindle pulleys, and a swashplate comprising one or more swashplate pulleys. A catheter can be coupled to the steering mechanism, the catheter having a proximal end, a distal end, and one or more catheter pull wires configured to control a movement of the distal end of the catheter, wherein the one or more catheter pull wires are routed through the one or more spindle pulleys and the one or more swashplate pulleys to couple the catheter to the steering mechanism. The steering mechanism with pulleys provides amplification between swashplate deflection and pull wire displacement.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This disclosure is related to PCT/US2023/078863, filed on Nov. 6, 2023, and U.S. Provisional No. 63/382,773, filed on Nov. 8, 2022, both of which are incorporated by reference herein in their entirety.

### TECHNICAL FIELD

The disclosure relates to catheter systems for controlling movement of a distal end of a catheter. More specifically, this disclosure relates an assembly that is coupled to catheter pull wires to move the distal tip of a catheter.

### BACKGROUND

Catheters are commonly used to access patient anatomy for medical reasons. Catheters are often exposed to biomaterials including bodily fluids, tissue, and pathogens. Accordingly, catheters are typically single-use devices to be disposed of after each use to prevent the transmission of biomaterials from one patient to another. Due to their single-use nature and to minimize costs, catheters are often constructed from inexpensive materials with simple mechanical elements for providing controls and lack sophisticated electrical components and control systems.

Control and movement (e.g., bending) of a distal end of a catheter is typically done by rotational movement of a dial or another type of actuator on a catheter handle coupled to the catheter that bends the distal tip in a direction (e.g., within a plane, or in three dimensions relative to an orientation of the catheter handle. However, such implementations do not allow rotation of the catheter without changing the direction of the bend of the distal tip. Accordingly, it would be advantageous to provide a catheter steering mechanism that allows a particular bend in a distal tip of a catheter while allowing the catheter, as a whole, to rotate. This can be useful for many purposes, including imaging. In addition, it may be advantageous to have an assembly in a catheter handle for moving the distal tip of a catheter that has a ratio of movement that provides for a small movement of a controller to result in a larger movement of the distal tip.

### SUMMARY

Certain aspects of this invention are defined by the independent claims. The dependent claims concern optional features of some embodiments of the invention. The systems, methods, and devices described herein each have several aspects, no single one of which is solely responsible for its desirable attributes. Without limiting the scope of this disclosure, several non-limiting features will now be discussed briefly.

In one aspect of the system and methods disclosed herein, catheter steering mechanism is described. The catheter steering mechanism includes a spindle, a swashplate, a catheter, and one or more steering wires. The spindle includes one or more spindle pulleys. The swashplate includes one or more swashplate pulleys. The catheter includes a proximal end, a distal end, and one or more catheter pull wires. The one or more catheter pull wires are configured to control a movement of the distal end of the catheter. The one or more catheter pull wires are routed through the one or more spindle pulleys and the one or more swashplate pulleys. The one or more steering wires attached to the swashplate, the one or more steering wires are configured to move the swashplate to a deflection angle thereby placing tension on the one or more catheter pull wires through the one or more spindle pulleys and the one or more swashplate pulleys and moving the distal end of the catheter to an amplified deflection angle.

The above and other aspects have various embodiments. For example, in some embodiments, the one or more spindle pulleys includes a first spindle pulley and a second spindle pulley. In some embodiments, the one or more catheter pull wires are routed under the first spindle pulley towards the second spindle pulley and then over the second spindle pulley towards the one or more swashplate pulleys. In some embodiments, the swashplate comprises a first swashplate pulley. In some embodiments, the one or more catheter pull wires are routed under the first spindle pulley towards the second spindle pulley, over the second spindle pulley towards the first swashplate pulley, and under the first swashplate pulley towards the spindle. In some embodiments, a proximal end of the one or more catheter pull wires is attached to the spindle. In some embodiments, the spindle further comprises a catheter receiving port disposed at the center of a top surface of the spindle. In some embodiments, the one or more catheter pull wires are routed through the catheter receiving port to the one or more spindle pulleys and the one or more swashplate pulleys.

In another aspect, a catheter steering mechanism includes a spindle, a swashplate, a catheter, and one or more steering wires. The spindle includes a first spindle pulley and a second spindle pulley. The swashplate includes a swashplate pulley. The catheter includes a proximal end, a distal end, and one or more catheter pull wires. The one or more catheter pull wires are configured to control a movement of the distal end of the catheter. The one or more catheter pull wires are routed under the first spindle pulley towards the second spindle pulley, over the second spindle pulley towards the first swashplate pulley, and under the first swashplate pulley towards the spindle. Theone or more steering wires are attached to the swashplate. The one or more steering wires are configured to move the swashplate to a deflection angle thereby placing tension on the one or more catheter pull wires through the first spindle pulley, the second spindle pulley, and the swashplate pulley and moving the distal end of the catheter to an amplified deflection angle.

The above and other aspects have various embodiments. For example, in some embodiments, the spindle further comprises a catheter receiving port disposed at the center of a top surface of the spindle, and wherein the one or more catheter pull wires are routed through the catheter receiving port to the first spindle pulley.

In another aspect, a catheter steering mechanism includes a spindle, a swashplate, a catheter, and one or more steering wires. The swashplate includes one or more pulleys. The catheter includes proximal end, a distal end, and one or more catheter pull wires. The one or more catheter pull wires are configured to control a movement of the distal end of the catheter, wherein the one or more catheter pull wires are routed through the one or more pulleys. The one or more steering wires are attached to the swashplate. The one or more steering wires are configured to move the swashplate to a deflection angle thereby placing tension on the one or more catheter pull wires through the one or more spindle pulleys and moving the distal end of the catheter to an amplified deflection angle.

In another aspect, a catheter steering mechanism includes a spindle, a swashplate, a catheter, and one or more steering wires. The spindle includes one or more spindle guiding surfaces. The swashplate includes one or more swashplate guiding surfaces. The catheter includes a proximal end, a distal end, and one or more catheter pull wires. The one or more catheter pull wires are configured to control a movement of the distal end of the catheter, wherein the one or more catheter pull wires are routed over the one or more spindle guiding surfaces and the one or more swashplate guiding surfaces. The one or more steering wires are attached to the swashplate. The one or more steering wires are configured to move the swashplate to a deflection angle thereby placing tension on the one or more catheter pull wires through the one or more spindle guiding surfaces and the one or more swashplate guiding surfaces and moving the distal end of the catheter to an amplified deflection angle.

### BRIEF DESCRIPTION OF THE DRAWINGS

FIG. 1A is a perspective view of an example of an ICE catheter handle, illustrating various structural and functional features of some embodiments.

FIG. 1B is a side view of the example of the embodiment of the ICE catheter handle illustrated in FIG. 1, and illustrating that the handle is configured to be coupled to a cap, and the cap is configured to be coupled to a catheter.

FIG. 2 is a perspective view of an example of another embodiment of a durable (reusable) catheter handle, which can be used with an ICE catheter, a IVUS catheter, an RF ablation catheter, and a FFR catheter, in various implementations.

FIG. 3 is a cross-sectional view of the catheter handle 1 taken along the plane 72 in FIG. 1, illustrating an embodiment of an assembly to move the distal tip of a catheter, the assembly including a swashplate that is my mechanical interaction with a plurality of extended members that move the swashplate in response to movement of a control on the catheter handle, where pull wires of a catheter are coupled to the swashplate.

FIG. 4 is a side view of another example of an assembly including a swashplate, where pull wires of a catheter are coupled to the swashplate that includes one or more pulleys, and where the pull wires extend from the catheter to the swashplate via the one or more pulleys.

FIG. 5 is a cross-sectional side view of an example of the swashplate illustrated in FIG. 4; and

FIG. 6 is a side view of an example of a pulley-less catheter system.

### DETAILED DESCRIPTION

The disclosure describes embodiments relating to a catheter system and methods for precise control of a catheter. More specifically, the embodiments relate to a handle device that may be used to control and articulate a catheter. The embodiments described herein may be used to perform ultrasound imaging using an Intra Cardiac Echography (“ICE”) catheter. The handle device may be used to control and articulate the ultrasound imaging device at a distal end of the catheter. The disclosure describes an example of an embodiment of a catheter steering mechanism which incorporates a swashplate to transmit forces from the steering handle input to the catheter pull wire output. The incorporation of a swashplate allows rotation of the catheter shaft and pull wires relative to the handle. When the swashplate is deflected, the catheter tip is bent according to the amount of deflection. When the deflected swashplate is rotated relative to the steering handle, it causes the catheter tip to rotate about its own center axis while maintaining its bend magnitude and orientation relative to the world reference frame. This is a valuable movement for steering the imaging plane of an ultrasound transducer.

The swashplate is deflected to transmit a displacement to the catheter pull wires. The swashplate is manipulated and held in position by a separate handle mechanism (not pictured). The swashplate design allows for the catheter/swashplate assembly to be rotated relative to the handle mechanism, causing the catheter to rotate about its own axis.

The addition of a pulley system provides more maneuverability and extends the range of motion of the catheter. The pulley system can also offer increased control of the catheter. Instead of the pull wires terminating on the swashplate itself, they are routed through a pulley on the swashplate and terminated on the spindle. Through this arrangement, the displacement of the pull wire is amplified. In the case of the pulley path shown in the figure, the pull wire displacement at a given deflection angle is approximately doubled compared to the original case of the pull wire terminating on the swashplate. More amplification could be achieved with additional pulleys and wraps of the pull wire (as in a block and tackle arrangement).

The chief advantage of the pulley mechanism is in reducing the amount of swashplate deflection needed to achieve a certain catheter tip deflection. Smaller swashplate angles in turn allow for a smaller overall mechanism footprint and reduction of undesired forces and non-linearities associated with higher angles.

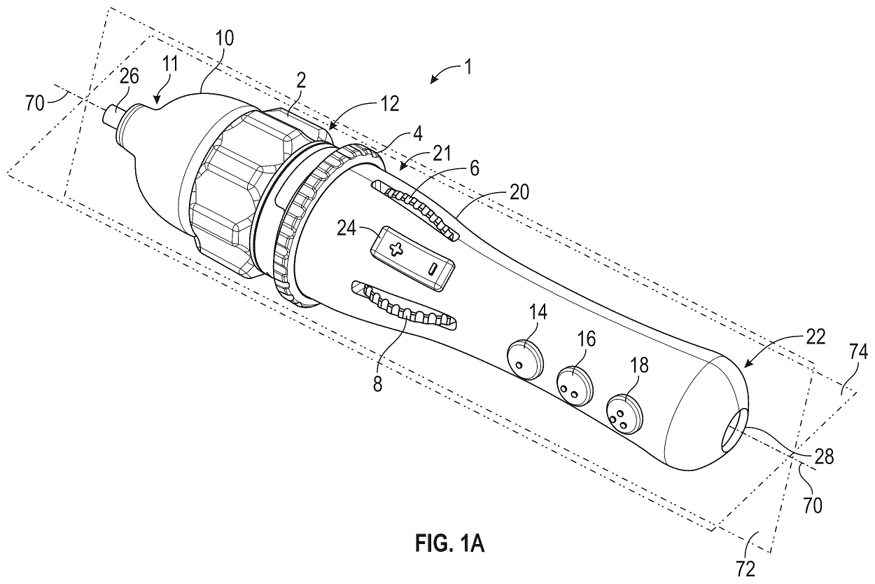

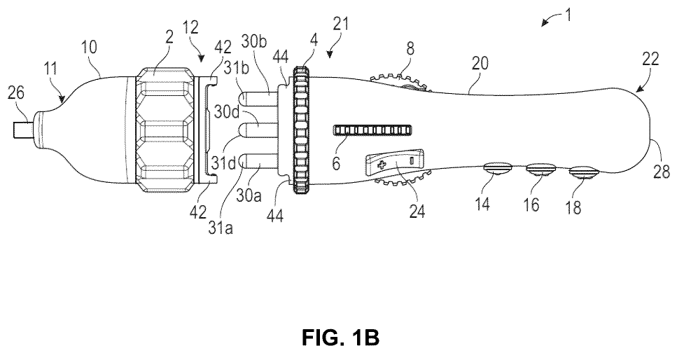

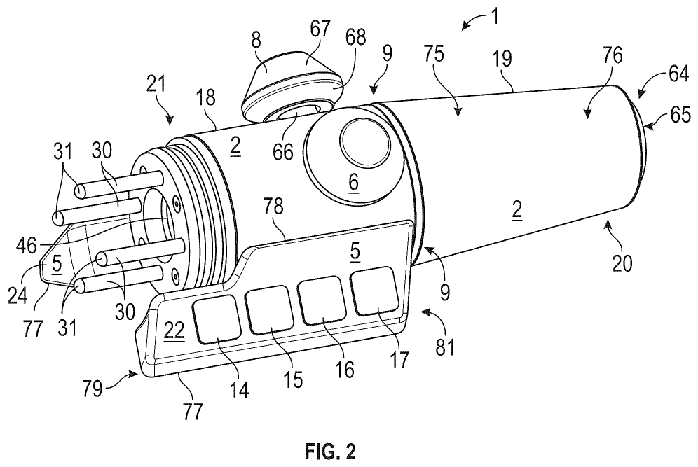

FIGS. 1A and 1B illustrate one embodiment of a catheter handle that may be configured to work with multiple catheters but can have some limitations for doing so. FIG. 2 illustrates another embodiment of a catheter handle configured to work with multiple types of catheters, and various structural and functional features of some embodiments. Specifically, FIG. 1A is a perspective view of an example of a catheter handle 1, illustrating various structural and functional features of some embodiments. FIG. 1B illustrates a different view of the catheter handle in FIG. 1A. The illustrated embodiments can be a durable (e.g., reusable) catheter handle. Additional information on the embodiments shown in FIGS. 1A and 1B can be found in U.S. application Ser. No. 17/820,139, filed Aug. 16, 2022, which is incorporated by reference herein in its entirety.

Although the examples of the catheter handle 1 may sometimes referred to as an ICE catheter handle because the functionality provided by the components of the catheter handle are advantageous for use with an ICE catheter, other types of catheters can also be used with the disclosed examples of catheter handles. In an example, the disclosed handles can be used with a catheter configured to perform intracardiac echocardiography (ICE), a catheter configured to perform intravascular ultrasound (IVUS) catheter, a catheter configured to perform radiofrequency (RF) ablation catheter, or a catheter configured to perform fractional flow reserve (FFR). In addition, the disclosed handles can be used with multi-mode catheters. For example, a catheter that is configured to perform ICE and ablation. In another example, a catheter that is configured to perform RF ablation and FFR. In other example, the catheter handle can be used with a catheter that performs any two or more of ICE, IVUS, ablation, or FFR. A catheter that perform two or more functions can be advantageous as it minimizes the invasiveness of having multiple catheters in the patient's vascular system and the heart.

In the embodiment illustrated in FIG. 1A, the catheter handle 1 includes a cap 10 which is a distal portion of the catheter handle 1, and a handle end 20 which is a proximal portion of the catheter handle 1. The outside surface of the cap 10 is sometimes referred to herein as the cap housing. The outside surface of the handle end 20 is sometimes referred to as the handle housing (or simply housing). Herein, “distal” refers to the portion of the catheter handle 1 that is closest to the catheter 26 (shown only in part) and to the patient when the catheter handle 1 is used in a medical procedure. The cap 10 includes a distal end 11 and a proximal end 12. The handle end 20 also includes a distal end 21 and a proximal end 22. The handle end 20 further includes a first actuator 6 (or thumbwheel 6), and a second actuator 8 (or thumbwheel 8). The handle end can also include one or more controls of various types, for example, a rocker switch 24 and three buttons 14, 16, 18. The handle end 20 includes a connector port 28 for connecting the catheter handle 1 to computing equipment (e.g., ultrasound processing equipment, a display, and the like). In this example, the catheter handle 1 also includes a rotation collar 2 aligned perpendicular to a longitudinal axis 70 of the catheter handle 1, for example, such that the axis of the rotation collar 2 aligns with the longitudinal axis 70. The rotation collar 2 is coupled to a catheter 26 at the proximal end of the catheter 26. The distal end 41 of the catheter 26 can include an ultrasound array for generating ICE images. The rotation collar 2 is configured to rotate around the longitudinal axis 70 such that a rotational movement of the rotation collar 2 rotates the catheter 26. The catheter handle 1 also includes a locking ring 4 is positioned on the proximal end 21 of the handle end 20 and perpendicularly aligned to the longitudinal axis 70. The locking ring 4 is configured to rotate around the longitudinal axis to lock a position of the catheter in a certain alignment/position.

In FIG. 1A, the ICE catheter handle 1 has a longitudinal axis 70 coincident with the center line if the catheter 1 and going from the distal end 11 of the cap 10 to the proximal end 22 of the handle end 20. Two planes 72, 74 that are orthogonal to each other are superimposed to the catheter handle 1 for illustration purpose. The plane 72 is the vertical plane in FIG. 1A, which passes through the first actuator 6 and is coincident with the axis 70 and the mid-plane of the first actuator 6. And the plane 74 is the horizontal plane in FIG. 1A, which passes through the second actuator 8 and is coincident with the axis 70 the mid-plane of the second actuator 8. Since the planes 72, 74 are orthogonal to each other, the first actuator 6 and the second actuator 8 are also orthogonally disposed in the handle 1.

FIG. 1B is a side view of the example of an ICE catheter handle 1 illustrated in FIG. 1A, showing that the handle 1 is configured to be coupled to a cap 10 which is connected to a catheter 26. The cap 10 may be removably coupled to the handle end 20 such that the cap 10 can be easily separated from, and coupled to, the handle end 20. The handle end 20 includes the locking ring 4 which is configured to rotate around the longitudinal axis 70 to realize locking the handle end 20 with the cap 10. The locking mechanism includes locking edges 40 on the cap 10 and locking recesses 44 on the handle end 20. When the handle end 20 is put together with the cap 10, the locking ring 4 is rotate to engage the locking edges 42 with the locking recesses 44, so that the handle end 20 is firmly connected to the cap 10. To disconnect the handle end 20 from the cap 10, the locking ring 4 is rotated to disengage the locking edge 42 and locking recesses 44.

In this example, the handle end 20 also includes control buttons 14, 16, and 18, as shown in FIG. 1A, that can be used to control various functions of the catheter, including imaging functions. In some embodiments, control buttons 14, 16, and 18 are programmable. The handle end 20 also includes an imaging control rocker switch 24 that controls a zoom feature of an ultrasound array on the catheter. The handle end 20 further includes a connection port 28 to connect the handle end 20, and the catheter attached to the handle end 20, to processing equipment, for example, image processing equipment to process and display ultrasound information generated by the catheter.

FIG. 2 is a perspective view of an example of an embodiment of a durable (reusable) catheter handle 1, which can be used with an ICE catheter, a IVUS catheter, an RF ablation catheter, and a FFR catheter, or various combinations of an ICE catheter, a IVUS catheter, an RF ablation catheter, and a FFR catheter, in various implementations.

In this example, catheter handle 1 has a housing 2 that has a proximal end 20 and a distal end 21. The catheter handle 1 includes a body portion (“body”) 18 on the distal end 21 and a tail portion (“tail”) 19 on the proximal end 20. The body 18 is coupled to a base 5. The base 5 is structured to prevent the catheter handle 1 from rotating when the catheter handle is set on a surface (e.g., a portion of a patient). In this example, the base 5 includes a first base portion 22 in the second base portion 24. The body 18, being coupled to the base 5, is non-rotatable relative to the base 5.

The catheter handle 1 further includes a first actuator 6 and a second actuator 8 positioned on the body 18 approximately 90 degrees apart. Similar to the actuators 6, 8 in FIG. 1A, the first and second actuators 6, 8 in FIG. 2 are coupled to mechanical assemblies that move pushrods 30 to control the angle of the swash plate 32 in the cap 10, which is connected to the catheter 26. In this embodiment, the first and second actuators 6, 8 are rotating knobs, each controlling a pair of oppositely positioned pushrods 30. Because this embodiment of the catheter handle includes an aperture 4 that runs throughout the housing 2, the mechanical assemblies that are coupled first and second actuator 6, 8 and the pushrods 30 are positioned between the aperture 4 and the outside surface of the housing 2. As illustrated in FIG. 2, each actuator 6, 8 includes an actuator shaft 66 that extends from mechanical assembly positioned inside the housing 2, through the housing 2. The actuator shaft 66 is coupled to a proximal portion 68 of an actuator, and a distal portion 67 of the actuator is coupled to the proximal portion 68. The proximal portion 68 and the distal portion 67 can be removably coupled together, for example, by using magnets. This allows a use case where the catheter handle 1 can be within a sterilized sleeve (or other material) with the proximal portion 68 also within the sterilized sleeve, and the distal portion 67 positioned on the outside of the sleeve and coupled to the proximal portion 68 such that the actuators 6, 8 can be easily used while the catheter handle is within the sterilized sleeve.

The base 5 includes an upper portion 78 that is coupled to the body 18 and a lower portion 78 that is configured to contact surface and in such a position holds a portion of the catheter handle stationary such that the catheter handle 1 as a whole does not rotate. Various configurations/designs of such a base 5 are contemplated where a base 5 is coupled to a portion of the catheter handle that does not rotate. In an example, the base 5 is coupled to a housing portion 2 of the catheter handle and they are configured such that the base 5 and the housing portion 2 do not move relative to each other. In the illustrated embodiment, the base 5 includes a first portion (or leg) 22 and a second portion (or leg) 24 that are positioned along the length of the body 18 and extend past the body 18 at the distal end 21 of the catheter handle to provide stability when the catheter handle/base is placed on a surface. Typically, when in use, catheter handle/base can be placed on a portion of the patient (for example, a leg of the patient) and the base being configured to have the first portion and the second portion can increase the stability when the surface is not flat (e.g., at least slightly cylindrical). In this embodiment, the base includes one or more controls (e.g., control buttons 14, 15, 16, and 17 in this example) which provide easily accessible controls (buttons, switches, etc.) for use during various catheter procedures. In some embodiments, the functionality of the controls are predetermined, while in other embodiments the functionality of the controls can be changed based on the user's preference for the procedure that is being performed. In other embodiments, the base 5 and/or the housing 2 can include more or fewer controls, different types of buttons/controls, and/or buttons/controls positioned in a different arrangement. By including buttons/controls on the base and/or a portion of the catheter handle that is intended to be oriented in a certain position during use, the apparatus can be easier to use because the controls are always in the same place, which can increase safety, efficiency, and speed of use especially after a medical practitioner is trained on the apparatus and uses it over time. Programmable controls can advantageously allow a medical practitioner to configure the controls to their own preference, in light of their way of performing a procedure and/or which controls are used more frequently or less frequently.

The tail 19 is rotatable, relative to the body 18 and the base 8, around the longitudinal axis 70. Typically, the tail 19 is typically rotated to an extent around the longitudinal axis 70 but is not rotated 360°. For example, the tail 19, and corresponding connected catheter, may be rotated in the range of about 0.1-180 degrees clockwise or counterclockwise. Typically, the tail 19 can be rotated between about 1° and 90°, between about 1° and 45°, between about 1°and 30°, between about 1° and 15°, or between about 1° and 7.5° (or less). An interface 9 is positioned between the rotatable tail 19 and the body 18. The interface 9 can include various structures that facilitate controlled movement of the tail 19 relative to the body 18 and locking the position of the tail 19 relative to the body 18. The tail 19 includes a distal surface 75 and the proximal surface 76. In some embodiments, the distal surface 75 is generally cylindrical. In some embodiments, the proximal surface 76 is generally oval which can facilitate rotation of the tail 19. The tail 19 is coupled to the pushrods 30 and other coupling structure at the distal end 21 of the catheter handle which is used to attach a catheter to the catheter handle. Accordingly, when the tail 19 is rotated around the longitudinal axis 70, the pushrods 30 and other controller mechanisms coupled to the actuators 6, 8, the coupling structure that attaches to the catheter, and a catheter attached to the catheter handle also rotates, while the body 18 and the base 5 do not rotate. The core is mechanically and electrically coupled to the tail 19, and to the catheter, and the core also rotates when the tail 19 is rotated.

The tail 19 includes, at the proximal end 20, an electrical interface 64 which is configured to be electrically connected to an electrical interface of the core when the core is inserted into an internal aperture of the housing 2 via opening 65 on the proximal end 20. The electrical interface 64 is electrically connected to the control buttons 14, 15, 16, and 17, and provides for communication of a signal from a control button to the control system 134, the control system 134 then controlling a function (imaging, ablation, sensing, etc.) of a catheter based on the signals communicated from the control button.

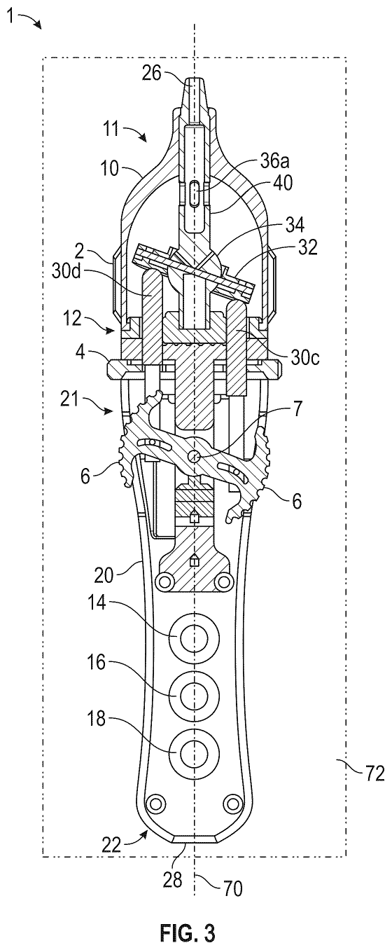

FIG. 3 illustrates an example of a cross-sectional view of the ICE catheter handle 1 in FIG. 1 taken along line plane 70, some internal structures in the device are revealed. As can be seen in FIG. 3, in this example the first actuator 6 is coupled with the body of the handle end 20 at a pivot 7 for the actuator 6. Two pushrods 30c, 30d can be disposed parallel to the axis 70 and slidably coupled to the first actuator 6 at an equal distance from the center of the actuator 6 at one end and in touch with a swash plate 32 at the other end. When the actuator 6 is rotated about the pivot 7 by the user's thumb or finger, the action forces one of the pushrods to move in a direction aligned with the axis 70 pressing against the swash plate 32 and the other pushrod to move in a direction aligned with the axis 70 but away from the swash plate 32, depending on which direction the actuator 6 is rotated.

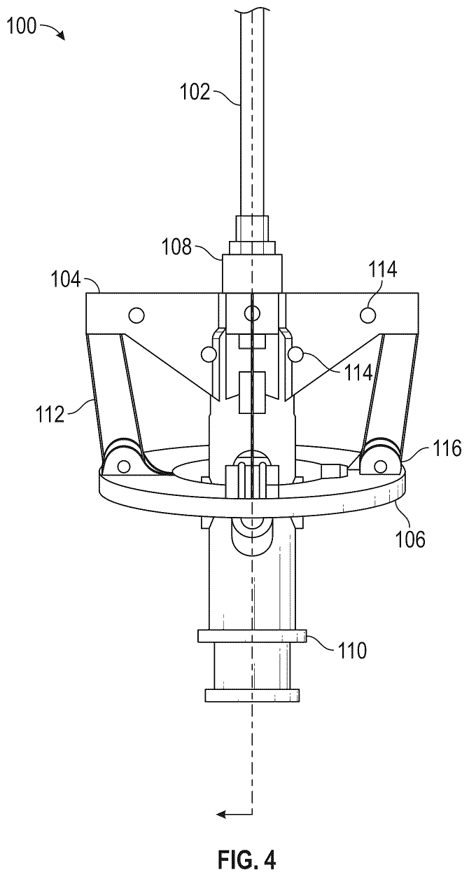

FIG. 4 is a lateral view of an example catheter system 100. The catheter system 100 includes a catheter 102, a spindle 104, and a swashplate 106. The spindle 104 and the swashplate 106 may be machined or molded parts. In some embodiments, the spindle 104 and the swashplate 106 may be sterilizable. The spindle 104 and the catheter 102 may be securely connected to one another at a catheter receiving port 108. In some embodiments, the catheter 102 may be maintained in a perpendicular position relative to a surface of the spindle 104. In some embodiments, the catheter system 100 may include an attachment mechanism 110 to receive a catheter control device (not pictured). For example, the attachment mechanism 110 may be a flange.

The catheter 102 may include one or more catheter pull wires 112. In some embodiments, as shown in FIG. 4, the one or more catheter pull wires 112 may be routed through a body of the catheter 102 to the spindle 104 and pass through the catheter receiving port 108. The one or more catheter pull wires 112 may then pass through one or more spindle pulleys 114, through one or more swashplate pulleys 116, and terminate at the spindle 104, where the one or more catheter pull wires 112 are attached. In some embodiments, the one or more catheter pull wires 112 may be routed through more or fewer components. The one or more catheter pull wires 112 may also be routed through the catheter 102, the spindle 104, the one or more spindle pulleys 114, and the one or more swashplate pulleys in different orders.

In some embodiments the one or more spindle pulleys 114 and the one or more swashplate pulleys 116 may be non-rotational. For example, the one or more spindle pulleys 114 and the one or more swashplate pulleys 116 may be stationary bosses that receive the one or more catheter pull wires 112. The one or more catheter pull wires 112 may freely pass over the surface of the one or more spindle pulleys 114 and the one or more swashplate pulleys 116, but the one or more spindle pulleys 114 and the one or more swashplate pulleys 116 do not move or rotate. In some embodiments, the spindle 104 and/or the swashplate 106 may include guiding surfaces that can be employed to route the one or more catheter wires 112 through the spindle 104 and the swashplate 106. The guiding surfaces may be employed in place of the one or more spindle pulleys 114 and the one or more swashplate pulleys 116.

The swashplate 106 may be connected to a catheter control device. The catheter control device may include one or more steering wires that are attached to the swashplate 106. The one or more steering wires may move, or rotate, the swashplate 106 within a housing. In some embodiments, the swashplate 106 may be an annular member that includes a curved inner surface configured to receive a ball member. The swashplate 106 may be disposed around a ball of ball joint. The swashplate 106 may be moved around the ball by placing tension on the one or more steering wires. In some embodiments, the swashplate 106 may be placed in a swashplate housing that allows the swashplate 106 to rotate within a particular range of motion. For example, the swashplate 106 may be an annular member, and the swashplate housing may include a spherical inner chamber that the swashplate moves within. The spherical chamber may have an opening on a top side and an opening on a bottom side to allow the steering wires and/or the one or more catheter wires to attach to the swashplate 106 or interact with the one or more swashplate pulleys 116.

During a procedure, the catheter control device may move the swashplate 106 with the one or more steering wires such that the swashplate 106 moves to a deflection angle. By moving the swashplate 106, tension is placed on the one or more catheter pull wires 112 via the one or more swashplate pulleys 116, and the distal end of the catheter 102 is moved by the one or more catheter pull wires. The one or more spindle pulleys 114 and the one or more swashplate pulleys 116 amplify the motion of the swashplate 106 such that the deflection angle of the distal end of the catheter 102 is a magnitude greater than the deflection of the swashplate. For example, the deflection angle of the distal end of the catheter 102 may be double the deflection angle of the swashplate 106.

In some embodiments, the deflection angle ratio between the swashplate 106 and a distal end of the catheter 102 may be determined based on an arrangement of the one or more spindle pulleys 114 and the one or more swashplate pulleys 116. For example, pulleys may be added or removed from the one or more spindle pulleys 114 or the one or more swashplate pulleys 116. The pulleys may also be rearranged to further increase the deflection angle ratio. For example, a block and tackle arrangement may be employed.

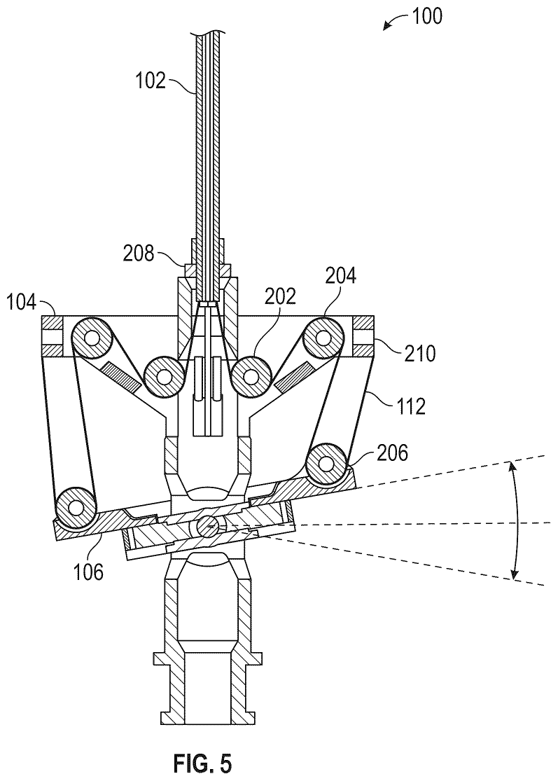

FIG. 5 is a lateral view of the catheter system 100. As discussed above in conjunction with FIG. 4, the catheter system includes a catheter 102, a spindle 104, and a swashplate 106. FIG. 5 further illustrates how the one or more catheter pull wires 112 may be routed through a first spindle pulley 202, a second spindle pulley 204, and a first swashplate pulley 206. The first spindle pulley 202 and the second spindle pulley 204 may be mounted within the spindle 104. For example, the first spindle pulley 202 may be located inside of the body of the spindle 104. In some embodiments, the spindle 104 may have a cavity or recess that houses the first spindle pulley 202 and/or the second spindle pulley 204. The first spindle pulley 202 may be disposed near a centerline axis of the spindle 104 and near a bottom surface of the spindle 104. The second spindle pulley 204 may be disposed further from the centerline of the spindle 104 than the first spindle pulley 202. The second spindle pulley 204 may also be disposed higher within the body of the spindle 104 than the first spindle pulley 202. For example, the second spindle pulley 204 may be located near a top surface of the spindle 104 such that an outer edge of the second spindle pulley 204 is coincident with the top surface of the spindle 104. In some embodiments, the first spindle pulley 202 and the second spindle pulley 204 may be mounted on a bottom surface or a top surface of the spindle 104. The first swashplate pulley 206 may be mounted on a top surface of the swashplate 106. In some embodiments, the first swashplate pulley 206 may be mounted within the body of the swashplate 106. The swashplate 106 may have a cavity or recess that receives the first swashplate pulley 206.

In some embodiments, as shown in FIG. 5, the one or more catheter pull wires 112 may be routed through a body of the catheter 102 to the spindle 104. The one or more catheter pull wires 112 may then pass through a catheter receiving port 208. The catheter receiving port 208 may be disposed at the center of the top surface of the spindle 104. After passing through the catheter receiving port 208, the one or more catheter pull wires 112 may be wrapped around the bottom of the first spindle pulley 202 and routed up toward the second spindle pulley 204. The one or more catheter pull wires 112 may then wrap around the top of the second spindle pulley 204 and be routed out from the bottom surface of the spindle 104, toward the first swashplate pulley 206. The one or more catheter pull wires 112 may wrap around the bottom of the first swashplate pulley 206 and be routed toward an outer edge of the spindle 104. The terminal end of the one or more catheter pull wires 112 may be attached to the outer edge of the spindle 104 at a point 210.

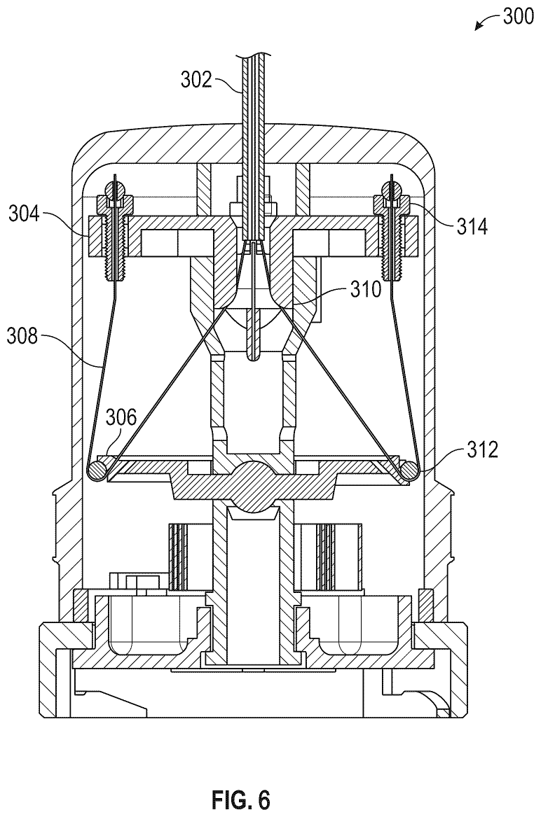

FIG. 6 is a lateral view of an example of a pulley-less catheter system 300. In some embodiments, elements of the catheter system 100 and the catheter system 300 may be combined. Similar to the catheter system 100, the catheter system 300 includes a catheter 302, a spindle 304, and a swashplate 306, and one or more catheter pull wires 308 may be routed through the catheter 302, spindle 304, and swashplate 306. The spindle 304 may include one or more spindle guiding surfaces 310, and the swashplate 306 may include one or more swashplate guiding surfaces 312. The one or more spindle guiding surfaces 310 and the one or more swashplate guiding surfaces 312 may be molded or embossed parts of the spindle 304 and the swashplate 306 respectively. In some embodiments, the one or more spindle guiding surfaces 310 and the one or more swashplate guiding surfaces 312 may be separate parts that are combined with the spindle 304 and the swashplate 306, respectively.

The one or more spindle guiding surfaces 310 and the one or more swashplate guiding surfaces 312 may route the one or more catheter pull wires 308 between the catheter 302, the spindle 304, and the swashplate 306. The one or more catheter pull wires 308 to routed over, under, through, or around the one or more spindle guiding surfaces 310 and the one or more swashplate guiding surfaces 312. In the example embodiments shown in FIG. 6, the one or more catheter pull wires 308 are routed through a body of the catheter 302 to the spindle 304. The one or more catheter pull wires 308 then pass over one or more spindle guiding surfaces 310 which direct the one or more catheter pull wires 308 toward the swashplate 306. The one or more catheter pull wires 308 are then routed through one or more swashplate guiding surfaces 312 and routed back toward the spindle 304 where the one or more catheter pull wires 308 may be anchored at an outer edge of the spindle 304. In some embodiments, the one or more swashplate guiding surfaces 312 may be round surfaces that resemble a pulley, and the one or more catheter pull wires 308 may be routed around the edge of the one or more swashplate guiding surfaces 312. In some embodiments, the one or more catheter wires 308 may be anchored to the spindle 304 via one or more screws 314. The one or more screws 314 may be configured to be moved to different radii on the spindle.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the embodiments should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached or coupled to the other feature or element or intervening features or elements may be present. In contrast, when a feature or element is referred to as being “directly connected”, “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a”, “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/”.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly”, “downwardly”, “vertical”, “horizontal” and the like may be used herein for the purpose of explanation only unless specifically indicated otherwise.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing numeric values of magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

What is claimed is:

1. A catheter steering mechanism, comprising:
a catheter comprising a proximal end, a distal end, and catheter pull wires configured to control a movement of the distal end of the catheter,
a spindle comprising one or more spindle pulleys;
a swashplate positioned in a plane perpendicular to the catheter and movable to a deflection angle, the swashplate comprising one or more swashplate pulleys, wherein the catheter pull wires are routed through the one or more spindle pulleys and the one or more swashplate pulleys and coupled to the spindle,
wherein moving the swashplate to a deflection angle places tension on the catheter pull wires through the one or more spindle pulleys and the one or more swashplate pulleys, moving the distal end of the catheter to an amplified deflection angle when compared to the deflection angle of the swashplate.

2. The catheter steering mechanism of claim 1, wherein the one or more spindle pulleys comprises a first spindle pulley and a second spindle pulley.

3. The catheter steering mechanism of claim 2, wherein one of the catheter pull wires is routed from the first spindle pulley to the second spindle pulley, and then to one of the one or more swashplate pulleys.

4. The catheter steering mechanism of claim 2, wherein the swashplate comprises a first swashplate pulley and a second swashplate pulley.

5. The catheter steering mechanism of claim 3, wherein the first spindle pulley and the second spindle pulley are positioned on the spindle on opposite sides of the spindle.

6. The catheter steering mechanism of claim 4, wherein the first swashplate pulley and a second swashplate pulley are positioned on the swashplate on opposite sides of the swashplate.

7. The catheter steering mechanism of claim 1, wherein the spindle further comprises a catheter receiving port disposed at the center of a top surface of the spindle.

8. The catheter steering mechanism of claim 1, wherein the swashplate is an annular member.

9. The catheter steering mechanism of claim 8, wherein the swashplate further comprises a curved inner surface configured to receive a ball member, wherein the swashplate is configured to moved around the ball member.

10. The catheter steering mechanism of claim 1, wherein the one or more spindle pulleys comprises a first spindle pulley and a second spindle pulley located in the spindle.

11. The catheter steering mechanism of claim 1, wherein the one or more spindle pulleys comprises a first spindle pulley and a second spindle pulley located in the spindle.

12. The catheter steering mechanism of claim 1, wherein the one or more spindle pulleys comprises a first spindle pulley and a second spindle pulley are mounted on a surface of the spindle.

13. A catheter steering mechanism, comprising:
a catheter comprising a proximal end, a distal end, and catheter pull wires configured to control a movement of the distal end of the catheter,
a spindle comprising a spindle pulley associated with each catheter pull wire; and
a swashplate positioned in a plane perpendicular to the catheter and movable to a deflection angle, the swashplate comprising a swashplate pulley associated with each catheter pull wire, each catheter pull wire routed through a spindle pulley and a swashplate pulley and coupled to the spindle,
wherein moving the swashplate to a deflection angle moves the distal end of the catheter, via the catheter pull wires, to an amplified deflection angle when compared to the deflection angle of the swashplate.

14. The catheter steering mechanism of claim 13, wherein the spindle comprises a first spindle pulley and a second spindle pulley positioned on the spindle on opposite sides of the spindle.

15. The catheter steering mechanism of claim 13, wherein the swashplate comprises a first swashplate pulley and a second swashplate pulley positioned on the swashplate on opposite sides of the swashplate.

16. The catheter steering mechanism of claim 13, wherein the spindle comprises a first spindle pulley and a second spindle pulley, and wherein the catheter pull wires comprise a first catheter pull wire, the first catheter pull wire routed on the first spindle pulley, the second spindle pulley, and a first swashplate pulley and then coupled to the spindle.

17. The catheter steering mechanism of claim 16, wherein the spindle comprises a third spindle pulley and a fourth spindle pulley, and wherein the catheter pull wires comprise a second catheter pull wire, the second catheter pull wire routed on the third spindle pulley, the fourth spindle pulley, and a second swashplate pulley and then coupled to the spindle.

18. A catheter steering mechanism, comprising:
a catheter comprising a proximal end, a distal end, and at least a first catheter pull wire and a second catheter pull wire configured to control movement of the distal end of the catheter,
a spindle comprising a first spindle pulley associated with the first catheter pull wire and a second spindle pulley associated with the second catheter pull wire; and
a deflectable swashplate positioned in a plane perpendicular to the catheter, the swashplate comprising a first swashplate pulley associated with the catheter pull wire and a second swashplate pulley associated with the second catheter pull wire, the first catheter pull wire routed on the first spindle pulley and the first swashplate pulley and then coupled to the spindle, the second catheter pull wire routed on the second spindle pulley and the second swashplate pulley and then coupled to the spindle,
wherein moving the swashplate to a deflection angle moves the distal end of the catheter via the catheter pull wires.

19. The catheter steering mechanism of claim 18, wherein the spindle further comprises a third spindle pulley associated with the first catheter pull wire and a fourth spindle pulley associated with the second catheter pull wire, wherein the first catheter pull wire is routed on the first spindle pulley, the third spindle pulley, and the first swashplate pulley and then coupled to the spindle, and the second catheter pull wire is routed on the second spindle pulley, the fourth spindle pulley, and the second swashplate pulley and then coupled to the spindle.
